(19) United States
(12) Patent Application Publication
(10) Pub. No.: US 2021/0059567 A1
(43) Pub. Date: Mar. 4, 2021

(54) SYSTEM AND METHOD FOR CORRECTING POSTURE BY USING SMART BAG

(71) Applicant: OTTHYANG CO., LTD., Daegu (KR)

(72) Inventors: Dong Hyun LEE, Daegu (KR); Ho Sook JANG, Daegu (KR)

(21) Appl. No.: 16/957,496

(22) PCT Filed: Oct. 5, 2018
(86) PCT No.: PCT/KR2018/011793

§ 371 Date: Jun. 24, 2020

(30) Foreign Application Priority Data
Dec. 27, 2017 (KR) 10-2017-0181414

(51) Int. Cl. A61B 5/11; A45F 3/04; A61B 5/00

(52) U.S. Cl. CPC A61B 5/1116; A45F 3/047; A61B 5/6802; A61B 2562/0247; A61B 5/6843; A61B 2560/0487; A61B 5/6831

(57) ABSTRACT

A system and a method for correcting posture are provided where the system comprises a smart bag including at least one pressure sensor and a first communication unit for transmitting an input value recognized by the pressure sensor to a user terminal. The user terminal including a second communication unit for receiving the input value from the first communication unit, an adhesion status checking unit for determining whether a user's human body and the smart bag are in close contact each other or not and determining an adhesion degree between the user's human body and the smart bag by using the input value received by the second communication unit, and a display unit for displaying the adhesion degree between the user's human body and the smart bag determined by the adhesion status checking unit.

### BACKGROUND

The present invention relates to a system and a method for correcting posture by using a smart bag, and more specifically, to a system and a method for correcting posture by using a smart bag that can correct the user's posture by determining the adhesion degree between a user's human body and a smart bag by using the smart bag and adjusting an auxiliary strap of the smart bag by using the determined adhesion degree.

When a user goes out or moves, a bag is used to store and carry various items. Generally, the bag is provided with a bag strap, so that the user can carry it on his shoulder. Such a bag strap can be adjusted by the user according to his physical condition.

In general, the bag strap includes a shoulder contact portion and a length adjusting portion, which are connected to each other by a connection buckle and coupled to the bag body. The users with various physical conditions can use the bag through a length adjustment. For example, when the user with a small physical condition uses the bag with a large body condition, the user reduces the length of the bag strap.

In this way, the users adjust the bag strap by roughly guessing the fit when the bag is worn. In this case, the user has a problem in that the length of the bag strap cannot be adjusted according to his physical condition. In addition, since it is difficult for the users to grasp the weight of items stored in the body of the bag, the users often carry items with excessive weight.

Due to this, there is a problem in that the weight of the bag is carried on the shoulder, causing a risk such as scoliosis.

In order to solve this problem, the conventional Korea Utility Model Registration No. 20-0309558 (registration on Mar. 24, 2003) discloses a bag strap capable of adjusting a length thereof.

In the case of such a bag strap that can be adjusted in length, it suggests a method of adjusting the length according to the physical condition using the bag sticker unit. However, since the user directly checks the bag sticker unit and manually adjusts it, there is uncertain whether it is suitable for the wearer's physical condition.

In addition, it cannot be said that such a bag sticker unit may have a positive effect on the user's posture correction.

### SUMMARY OF THE INVENTION

Accordingly, the present invention has been made in an effort to solve the problems and a first object of the present invention is to provide a system for correcting posture by using a smart bag in that it automatically adjusts the length of the bag string suitable for the user's human body condition, uses an image and the like obtained by capturing the body of the user and the like so as to determine the current posture of the user and, simultaneously and automatically, adjusts the length of a bag strap suitable for the current posture of the user, thereby helping correct the posture of the user.

In addition, a second object of the present invention is to provide a method for correcting posture by using a smart bag in that it automatically adjusts the length of the bag string suitable for the user's human body condition, uses an image obtained by capturing the body of the user and the like so as to determine the current posture of the user and, simultaneously and automatically, adjusts the length of a bag strap suitable for the current posture of the user, thereby helping correct the posture of the user.

In order to achieve the first object, the present invention provides a system for correcting posture including: a smart bag including at least one pressure sensor and a first communication unit for transmitting an input value recognized by the pressure sensor to a user terminal; and the user terminal including a second communication unit for receiving the input value from the first communication unit, an adhesion status checking unit for determining whether a user's human body and the smart bag are in close contact each other or not and determining an adhesion degree between the user's human body and the smart bag by using the input value received by the second communication unit, and a display unit for displaying the adhesion degree between the user's human body and the smart bag determined by the adhesion status checking unit.

The user terminal further may include a storage unit for storing a user's human body information, a posture and a length of an auxiliary strap matched to the user's human body information as well as the user's human body information; and a control unit for generating a first control signal for winding or unwinding the auxiliary strap of the smart bag by using adhesion information between the user's human body and the smart bag, wherein the second communication unit transmits the first control signal generated by the control unit to the first communication unit.

The user terminal may include an input unit for generating a second control signal to wind or unwind the auxiliary strap of the smart bag according to the user's intention, wherein the second communication unit transmits the second control signal generated by the input unit to the first communication unit.

The user terminal may include an input unit for inputting an image obtained by capturing the user's human body and at least one of first to sixth input signals; a posture determination unit for determining a posture of the user by using the image obtained by capturing the user's human body input by the input unit and at least one of the first to sixth input signals; a storage unit for storing the lengths of the auxiliary strap matched to each posture of the user; and a control unit for generating a third control signal to wind or unwind the auxiliary strap of the smart bag by using the lengths of the auxiliary strap matched to each posture of the user, wherein the second communication unit transmits the third control signal generated by the control unit to the first communication unit.

The user terminal may include a winding unit for winding or unwinding the auxiliary strap so as to have a length corresponding to the first control signal received by the first communication unit; and a motor unit for providing a driving force to the winding unit.

The user terminal may include a winding unit for winding or unwinding the auxiliary strap so as to have a length corresponding to the second control signal received by the first communication unit; and a motor unit for providing a driving force to the winding unit.

The user terminal may include a winding unit for winding or unwinding the auxiliary strap so as to have a length corresponding to the third control signal received by the first communication unit; and a motor unit for providing a driving force to the winding unit.

In order to achieve the second object, the present invention provides a method for correcting posture, including steps of: transmitting an input value recognized by at least pressure sensor to a second communication unit by means of a first communication unit; determining whether a user's body and a smart bag are in close contact or not, by using the input value received by the second communication unit, by means of an adhesion status checking unit; determining an adhesion degree between the user's human body and the smart bag by means of the adhesion status checking unit; and displaying the adhesion degree between the user's human body and the smart bag determined by the adhesion status checking unit by means of a display unit.

The step of determining the adhesion degree between the user's human body and the smart bag by means of the adhesion status checking unit may comprise steps of: generating a first control signal for winding or unwinding an auxiliary strap of the smart bag by means of a control unit, by using adhesion information between the user's human body and the smart bag determined by the adhesion status checking unit; transmitting the first control signal to the first communication unit by means of the second communication unit; and winding or unwinding the auxiliary strap by means of a winding unit so as to have a length corresponding to the first control signal received by the first communication unit.

The step of determining the adhesion degree between the user's human body and the smart bag by means of the adhesion status checking unit may comprise steps of: generating a second control signal to wind or unwind the auxiliary strap of the smart bag according to the user's intention by means of an input unit; transmitting the second control signal to the first communication unit by means of the second communication unit; and winding or unwinding the auxiliary strap by means of the winding unit so as to have a length corresponding to the second control signal received by the first communication unit.

The step of determining the adhesion degree between the user's human body and the smart bag by means of the adhesion status checking unit may comprise steps of: inputting an image obtained by capturing the user's human body and at least one of first to sixth input signals by means of an input unit; determining a posture of the user by means of a posture determination unit, by using the image obtained by capturing the user's human body and at least one of the first to the sixth input signals; generating a third control signal to wind or unwind the auxiliary strap of the smart bag by means of the control unit, by using the length of the auxiliary strap matched to each posture of the user stored by a storage unit; transmitting the third control signal to the first communication unit by means of the second communication unit; and winding or unwinding the auxiliary strap by means of the winding unit so as to have a length corresponding to the third control signal received by the first communication unit.

According to the system and the method for correcting a posture by using the smart bag as described above, there is an effect in that it automatically adjusts the length of the bag string suitable for the user's human body condition, uses the image obtained by capturing the body of the user and the like so as to determine the current posture of the user and, simultaneously and automatically, adjusts the length of a bag strap suitable for the current posture of the user, thereby helping correct the posture of the user.

The above and other objects, features and advantages of the present invention will be more apparent from the following detailed description taken in conjunction with the accompanying drawings.

### BRIEF DESCRIPTION OF THE INVENTION

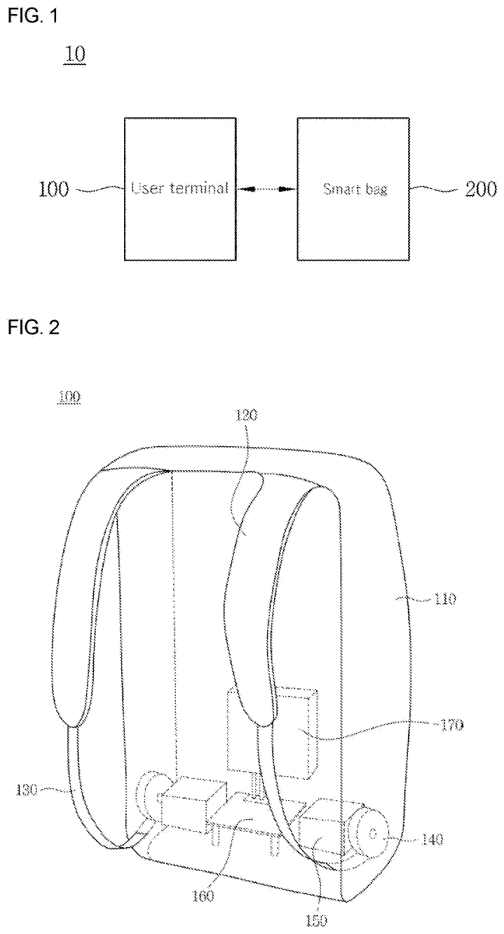

FIG. 1 is a view showing a schematic configuration of a system for correcting posture by using a smart bag according to an embodiment of the present invention;

FIG. 2 is a view showing a smart bag as one configuration of the present invention

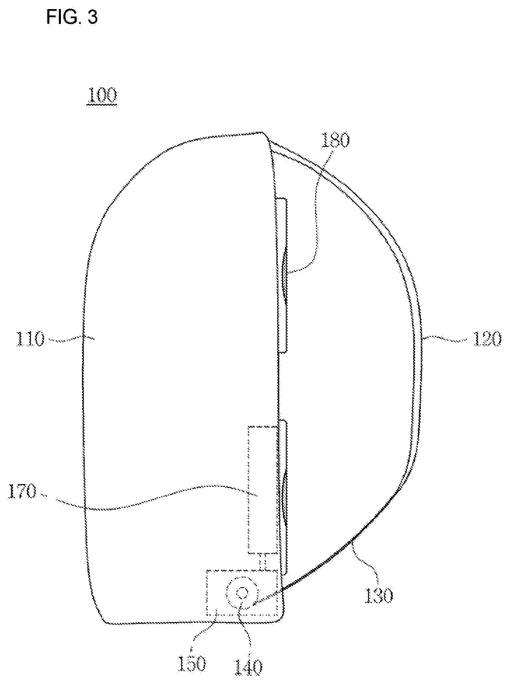

FIG. 3 is a side view showing a smart bag as one configuration of the present invention;

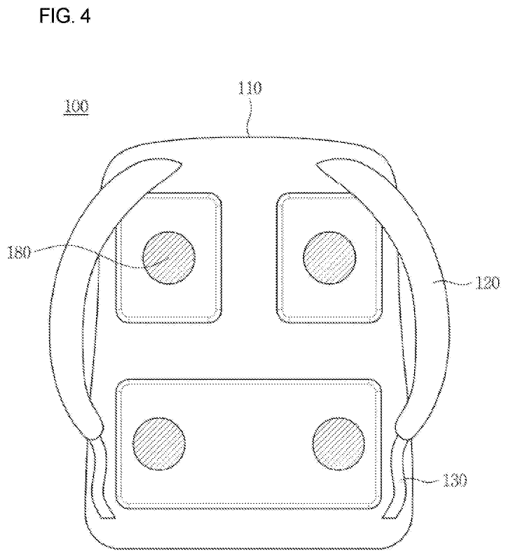

FIG. 4 is a rear view showing a smart bag as one configuration of the present invention;

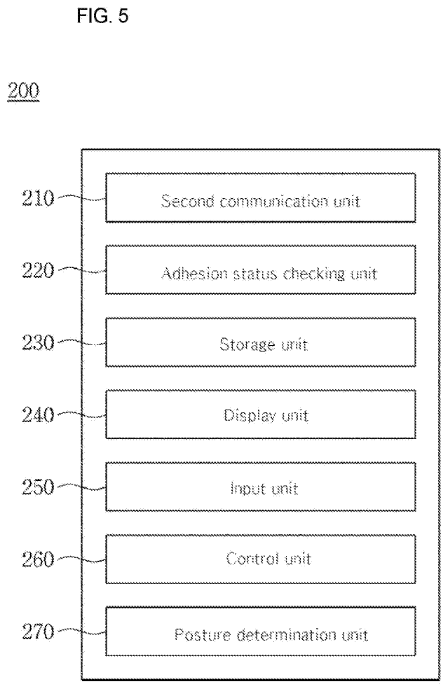

FIG. 5 is a view showing a schematic configuration of a user terminal as one configuration of the present invention;

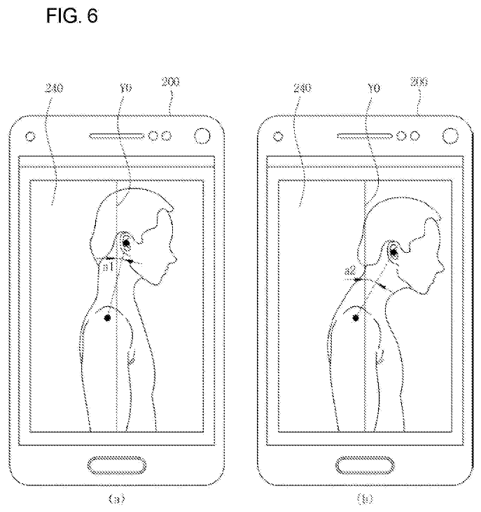

FIG. 6 is a diagram for explaining how to determine a forward head posture in accordance with an embodiment of the present invention;

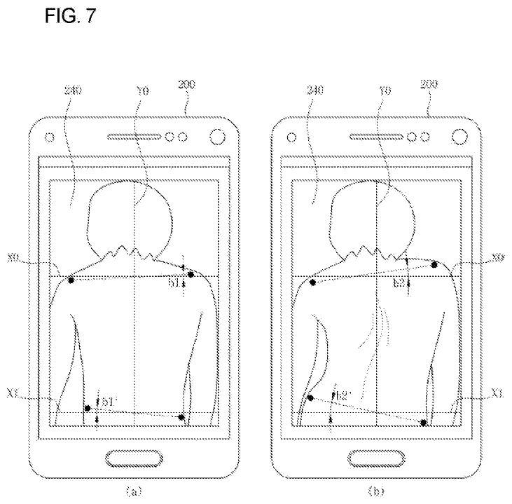

FIG. 7 shows a diagram for explaining how to determine scoliosis according to an embodiment of the present invention; and

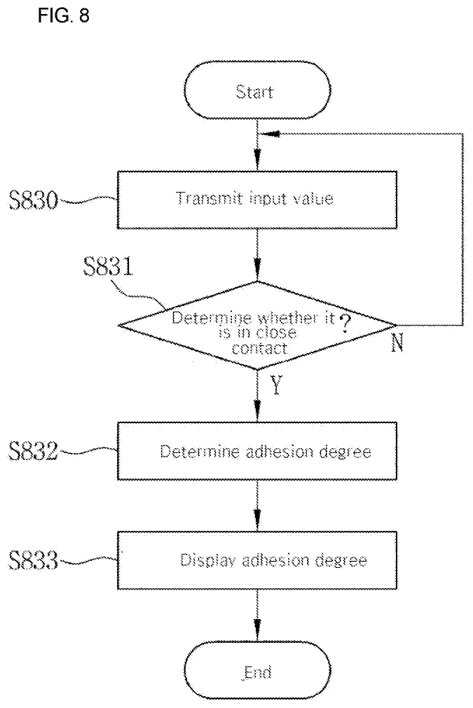

FIG. 8 is a view showing a schematic configuration of a posture correction method using a smart bag according to an embodiment of the present invention.

### DETAILED DESCRIPTION OF THE INVENTION

The terms or words used in the present specification and claims are not to be construed as being limited to ordinary or dictionary meanings. It should be interpreted as meanings and concepts consistent with the technical idea of the present invention based on the principle that the concept of terms can be properly defined in order to explain the invention in the best way.

Throughout the specification, when a part “includes” a certain component, it means that the component may further include other components, not to exclude other components, unless otherwise specified. In addition, terms such as “ . . . unit”, “ . . . group”, “module”, and “device” and the like described in the specification mean a unit that processes at least one function or operation and can be implemented as a combination of hardware and/or software.

Terms used in the embodiments of the present invention will be briefly described and the embodiments will be described in detail.

The terms used in the embodiments of the present invention are selected as general terms that are currently widely used as possible while considering the functions in the present invention. However, this may vary depending on the intention or precedent of a person skilled in the art or the appearance of new technologies etc. In addition, in certain cases, some terms are arbitrarily selected by the applicant, and in this case, their meanings will be described in detail in the description of the corresponding embodiments. Therefore, the terms used in the present embodiments should be defined based on the meaning of the terms and the contents of the present embodiments, not simply the names of the terms.

In an embodiment of the present invention, the terms such as “the first”, “the second” and so on are used to distinguish one element from another. For example, the first component may be referred to as a second component without departing from the scope of the present invention, and similarly, the second component may be referred to as a first component. The term called “and/or” includes the combination of the plural described and related items or a certain item of the plural described and related items.

In addition, in the embodiments of the present invention, the singular expression includes plural expressions unless it is apparently different in the context.

In addition, in the embodiments of the present invention, the terms such as “include”, “equipped” or “have” in this application intend to designate that the feature, number, stage, movement, component, part or the combination described in the specification exist. However, it should be understood that one or more other features or numbers, steps, actions, components, parts or combinations thereof are not excluded in advance.

In addition, in the embodiments of the present invention, ‘module’ or ‘unit’ performs at least one function or operation and may be implemented by hardware or software or a combination of hardware and software. In addition, a plurality of ‘modules’ or a plurality of ‘units’ may be integrated with at least one module except for a ‘module’ or a ‘unit’ that needs to be implemented with specific hardware to be implemented with at least one processor.

Further, in the embodiments of the present invention, also, when it is mentioned to be “connected” or “linked” to the other component, a certain component may be connected or linked to the other component. However, it will be understood that there may be some other components between them.

Hereinafter, embodiments of the present invention will be described in detail with reference to the accompanying drawings.

FIG. 1 is a view showing a schematic configuration of a system for correcting posture by using a smart bag according to an embodiment of the present invention, FIG. 2 is a view showing a smart bag as one configuration of the present invention, FIG. 3 is a side view showing a smart bag as one configuration of the present invention, FIG. 4 is a rear view showing a smart bag as one configuration of the present invention, and FIG. 5 is a view showing a schematic configuration of a user terminal as one configuration of the present invention.

Referring to FIG. 1, the system for correcting posture 10 includes a smart bag (100) and a user terminal (200).

Referring to FIG. 2 to FIG. 4, the smart bag (100) includes a bag body (110), a shoulder strap (120), an auxiliary strap (130), a winding unit (140), a motor unit (150), and a first communication unit (160), a power supply (170), and a pressure sensor (180).

The pressure sensor (180) may be one or more.

One or more pressure sensors (180) may be installed on one surface of the bag body (110) so that, when the user generally wears the smart bag (100), the sensors may be positioned on the back of the user.

Like this, when the user wears the smart bag (100), the adhesion degree between the user's human body and one surface of the smart bag (100) may be checked by using one or more pressure sensors (180).

The first communication unit (160) may transmit the pressure value recognized by one or more pressure sensors (180) to the user terminal (200).

In addition, the smart bag (100) includes a first communication unit (160) that transmits a pressure value recognized by one or more pressure sensors (180) to the user terminal (200).

The first communication unit (160) may be installed inside the bag body (110).

In the case of data transmission and reception between the first communication unit (160) and the user terminal (200), wired or wireless communication may be used.

Also, in a case of the wireless communication, various communication chips such as a Wi-Fi chip, a Bluetooth chip, an NFC chip, and a wireless communication chip may be used. At this time, the Wi-Fi chip, the Bluetooth chip, and the NFC chip perform communication using a WiFi method, a Bluetooth method, and an NFC method, respectively. Among them, the NFC chip means a chip that operates in a Near Field Communication (NFC) method using a 13.56 MHz band among various RF-ID frequency bands such as 135 kHz, 13.56 MHz, 433 MHz, 860 to 960 MHz, and 2.45 GHz. In a case of using the Wi-Fi chip or the Bluetooth chip, various connection information such as a SSID and a session key etc. may be first transmitted and received and then, communication information may be used to transmit and receive various information. The wireless communication chip refers to a chip that performs communication according to various communication standards such as IEEE, Zigbee, 3G (3rd Generation), 3GPP (3rd Generation Partnership Project), and LTE (Long Term Evoloution) etc.

One or more shoulder straps (120) maybe connected to one side of the bag body (110).

The auxiliary strap (130) maybe connected to one end of the shoulder strap (120) and one end of the auxiliary strap (130) may be connected to the winding unit (140) through the other side of the bag body (110).

In addition, the winding unit (140) may wind up or unwind the auxiliary strap (130) and the length of the auxiliary strap (130) can be adjusted through the winding unit (140).

More specifically, the winding unit (140) may wind or unwind the auxiliary strap (130) so as to have a length corresponding to a first control signal received by the first communication unit (160) and may wind or unwind the auxiliary strap (130) so as to have a length corresponding to a second control signal received by the first communication unit (160). In addition, the winding unit (140) may wind or unwind the auxiliary strap (130) so as to have a length corresponding to a third control signal received by the first communication unit (160).

Like this, in order to wind or unwind the auxiliary strap (130), that is, wind or untie the auxiliary strap (130) by means of the winding unit (140), a driving force is required. Here, the driving force may be provided by the motor unit (150).

That is, the motor unit (150) may provide the driving force to the winding unit (140).

Also, the power supply (170) may supply power to at least one of the first communication unit (160) and the motor unit (150).

In addition, the power supply (170) may transmit the battery residual quantity to the first communication unit (160).

At this time, the first communication unit (160) may transmit the battery residual quantity received from the power supply (170) to the user terminal (200).

Referring to FIG. 5, the user terminal (200) may include a second communication unit (210), an adhesion status checking unit (220), a storage unit (230), a display unit (240), an input unit (250), a control unit (260), and a posture determination unit (270).

The second communication unit (210) may receive a pressure value from the first communication unit (160).

In addition, the adhesion status checking unit (220) may determine whether the user's human body and the smart bag (100) are in close contact each other or not by using the pressure value received by the second communication unit (210).

That is, the adhesion status checking unit (220) compares the pressure value received by the second communication unit (210) with preset step-by-step pressure values, so that it can be determined whether the user's human body of currently wearing the smart bag (100) is in close contact with the smart bag (100) or not.

In addition, the adhesion status checking unit (220) compares the pressure value received by the second communication unit (210) with the preset step-by-step pressure values, so that it can determine the adhesion degree between the user's human body of currently wearing the smart bag (100) and the smart bag (100).

In addition, the display unit (240) may display the adhesion information between the user's human body and the smart bag (100) determined by the adhesion status checking unit (220).

In addition, the display unit (240) may display the battery residual quantity received by the second communication unit (210) from the first communication unit (160).

Through this, it is possible to easily check the battery residual quantity of the power supply (170).

The display unit (240) may include at least one display among a liquid crystal display, a thin film transistor-liquid crystal display, an organic light-emitting diode, a flexible display, and a three-dimensional display (3D display).

In addition, the control unit (260) uses the adhesion information between the user's human body and the smart bag (100), which is determined by the adhesion status checking unit (220), so that it can generate the first control signal for winding or loosening the auxiliary strap (130) of the smart bag (100).

The second communication unit (210) described above may transmit the first control signal generated by the control unit (260) to the first communication unit (160).

In addition, the input unit (250) may generate a second control signal to wind or unwind the auxiliary strap (130) of the smart bag (100) according to the user's intention.

Also, the input unit (250) may input images obtained by capturing the body of the user and at least one of first to sixth input signals.

The input unit (250) may include various input devices such as a touch input unit, a button, a voice input unit, a motion input unit, a keyboard, a mouse, etc. to receive a user command. Also, the input unit (250) may include a key pad, a dome switch, a touch pad (static pressure/constant voltage), a jog wheel, a jog switch, and the like.

In addition, the input unit (250) maybe a touch pad having a mutual layer structure with the display unit (240) and may be called as a touch screen.

In addition, when the touch pad of constituting the input unit (250) is configured as a touch screen by forming a mutual layer structure with the display unit (240), the display unit (240) may be used as an input device, that is, the input unit (250) in addition to the display device.

That is, the display unit (240) and the input unit (250) may be configured as one.

The touch screen may be configured to detect not only the touch input position and the touched area but also the touch input pressure. In addition, the touch screen may be configured to detect not only a touch (real-touch) but also a proximity touch.

In addition, the posture determination unit (270) may determine the posture of the user by using the image obtained by capturing the user's human body input by the input unit (250) and at least one of the first to sixth input signals.

In addition, the above-described control unit (260) may generate a third control signal to wind or unwind the auxiliary strap (130) of the smart bag (100) by using the auxiliary strap lengths matched to each posture of the user.

Here, the lengths of the auxiliary strap matched to each posture of the user may be stored in the storage unit (230) to be described later.

In the case of the length of the matching auxiliary strap for each user's posture, it refers to the length of the auxiliary strap matched to the user's forward head posture degree or the user's scoliosis degree.

In addition, the second communication unit (210) may transmit the second control signal generated by the input unit (250) to the first communication unit (160).

In addition, the second communication unit (210) may transmit the third control signal generated by the control unit (260) to the first communication unit (160).

In addition, the storage unit (230) may store the user's human body information and the posture and the length of the auxiliary strap matched to the user's human body information.

Here, the human body information of the user may include human body information of various people.

FIG. 6 is a diagram for explaining how to determine a forward head posture in accordance with an embodiment of the present invention and FIG. 7 shows a diagram for explaining how to determine scoliosis according to an embodiment of the present invention.

FIG. 6 is a diagram for explaining how to determine a forward head posture of the user by the posture determination unit (270). FIG. 6(a) is a case that the user is not a forward head posture and FIG. 6(b) is a case that the user is a forward head posture.

The posture determination unit (270) can determine whether the user is a forward head posture by using a preset vertical reference line (Y0), an image of the user's human body input by the input unit (250), a first input signal, and a second input signal.

More specifically, the input unit (250) may input a first input signal indicating a user's shoulder point and a second input signal indicating a user's ear point in the image of the user's human body.

In addition, the posture determination unit (270) may form a first straight line by connecting the first point where the first input signal is specified and the second point where the second input signal is specified.

As shown in (a) of FIG. 6, a first specific angle (a1) maybe formed by using the first straight line and the vertical reference line (Y0 formed by the posture determination unit (270). The posture determination unit (270) compares the preset reference angle of forward head posture with the first specific angle (a1). At this time, when the first specific angle (a1) is less than the preset forward head posture reference angle, it can determine that the user is not the forward head posture.

In addition, as shown in FIG. 6(b), the first specific angle (a2) may be formed by using the first straight line and the vertical reference line (Y0) formed by the posture determination unit (270). The posture determination unit (270) compares the preset forward head posture reference angle with the first specific angle (a2). At this time, when the first specific angle (a2) is greater than or equal to the preset forward head posture reference angle, it can determine that the user is the forward head posture.

In addition, the posture determination unit (270) may form the first specific angle (a2) by using the formed first straight line and the vertical reference line (Y0). Therefore, the posture determination unit (270) may compare the preset forward head posture reference angle with the first specific angle (a2) to determine the user's forward head posture degree.

FIG. 7 is a diagram for explaining how to determine a scoliosis of the user by the posture determination unit (270). FIG. 7(a) is a case that the user is not a scoliosis and FIG. 7(b) is a case that the user is a scoliosis.

The posture determination unit (270) can determine whether the user is a scoliosis by using a preset vertical reference line (Y0), a preset first horizontal reference line (X0), a preset second horizontal reference line (X1), an image of the user's human body input by the input unit (250), and a third to a sixth input signals.

More specifically, the input unit (250) may input a third input signal and a fourth input signal indicating the shoulder points on both sides of the user in the image of the user's human body.

In addition, the input unit (250) may input a fifth input signal and a sixth input signal indicating the side flank points of both sides of the user in the image of the user's human body.

In addition, the posture determination unit (270) may form a second straight line by connecting the third point where the third input signal is specified and the fourth point where the fourth input signal is specified for the shoulder points on both sides of the user in the image of the user's human body.

In addition, the posture determination unit (270) may form a third straight line by connecting the fifth point where the fifth input signal is specified and the sixth point where the sixth input signal is specified for the side flank points of both sides of the user in the image of the user's human body.

As shown in (a) of FIG. 7, a second specific angle (b1) may be formed by using the second straight line and the first horizontal reference line (X0) formed by the posture determination unit (270). The posture determination unit (270) compares the preset shoulder reference angle with the second specific angle (b1). At this time, when the second specific angle (b1) is less than the shoulder reference angle, it may be determined that the user's shoulder correction is not required.

In addition, a third specific angle (b1′) may be formed by using the third straight line and the second horizontal reference line (X1) formed by the posture determination unit (270). The posture determination unit (270) compares the preset flank reference angle with the third specific angle (b1′). At this time, when the third specific angle (b1′) is less than the flank reference angle, it may be determined that the user's flank correction is not required.

Also, as shown in (b) of FIG. 7, a second specific angle (b2) may be formed by using the second straight line and the first horizontal reference line (X0) formed by the posture determination unit (270). The posture determination unit (270) compares the preset shoulder reference angle with the second specific angle (b2). At this time, when the second specific angle (b2) is greater than or equal to the preset shoulder reference angle, it may be determined that the user's shoulder correction is necessary.

In addition, a third specific angle (b2′) may be formed by using the third straight line and the second horizontal reference line (X1) formed by the posture determination unit (270). The posture determination unit (270) compares the preset flank reference angle with the third specific angle (b2′). At this time, when the third specific angle (b2′) is greater than or equal to a predetermined side reference angle, it may be determined that the user's flank correction is necessary.

In addition, the posture determination unit (270) compares the preset shoulder reference angle with the second specific angle (b2), so that it can determine the degree of shoulder correction of the user, when the second specific angle (b2) is greater than or equal to the preset shoulder reference angle.

In addition, the posture determination unit (270) compares the preset flank reference angle with the third specific angle (b2′), so that it can determine the degree of flank correction of the user, when the third specific angle (b2′) is greater than or equal to the preset flank reference angle.

In addition, the posture determination unit (270) may determine whether the user is a scoliosis and determine the degree of the user's scoliosis, by using the degree of shoulder correction of the user and the degree of flank correction of the user.

In other words, at least one of the status of the user's forward head posture, the degree of the user's forward head posture, the degree of the user's shoulder correction, the degree of the user's flank correction, and the degree of the user's scoliosis determined by the posture determination unit (270) can be quantified.

In addition, the display unit (240) may display the determination process and results of the posture determination unit (270) described above.

That is, the display unit (240) may display the status of the user's forward head posture, the degree of the user's forward head posture, the degree of the user's shoulder correction, the degree of the user's flank correction, and the degree of the user's scoliosis determined by the posture determination unit (270) as well as the quantified contents thereof.

FIG. 8 is a view showing a schematic configuration of a posture correction method using a smart bag (100) according to an embodiment of the present invention.

Referring to FIG. 8, the first communication unit (160) may transmit the pressure value recognized by one or more pressure sensors (180) to the second communication unit (210). (S830)

In addition, the adhesion status checking unit (220) may determine whether the user's human body and the smart bag (100) are in close contact using the pressure value received by the second communication unit (210). (S831)

As a result of the determination of the adhesion status checking unit (220), when the user's human body and the smart bag (100) are not in close contact, the step 5830 may be performed.

In addition, as a result of the determination of the adhesion status checking unit (220), when the user's human body and the smart bag (100) are in close contact, a step 5832 may be performed.

As a result of the determination of the adhesion status checking unit (220), when the user's human body and the smart bag (100) are in close contact, the adhesion status checking unit (220) may determine the adhesion degree between the user's human body and the smart bag (100). (S832)

More specifically, the control unit (260) uses the adhesion information between the user's human body and the smart bag (100) determined by the adhesion status checking unit (220), so that it can generate the first control signal for winding or unwinding the auxiliary strap (130) of the smart bag (100).

In addition, the second communication unit (210) may transmit the first control signal to the first communication unit (160).

In addition, the winding unit (140) may wind or unwind the auxiliary strap (130) so as to have a length corresponding to the first control signal received by the first communication unit (160).

In addition, the input unit (250) may generate a second control signal to wind or unwind the auxiliary strap (130) of the smart bag (100) according to the user's intention.

Also, the second communication unit (210) may transmit the second control signal to the first communication unit (160).

In addition, the winding unit (140) may wind or unwind the auxiliary strap (130) to have a length corresponding to the second control signal received by the first communication unit (160).

Also, the input unit (250) may input the image obtained by capturing the user's human body and at least one of the first to the sixth input signals.

In addition, the posture determination unit (270) may determine the posture of the user by using the image of the user's human body and at least one of first to sixth input signals input by the input unit (250).

In addition, the control unit (260) may generate the third control signal to wind or unwind the auxiliary strap (130) of the smart bag (100) by using the auxiliary strap lengths matched to each posture of the user stored by the storage unit (230).

In addition, the second communication unit (210) may transmit the third control signal to the first communication unit (160).

In addition, the winding unit (140) may wind or unwind the auxiliary strap (130) so as to have a length corresponding to the third control signal received by the first communication unit (160).

In addition, the display unit (240) may display the adhesion degree between the user's human body and the smart bag (100) determined by the adhesion status checking unit (220). (S833)

As described above, the configuration and operation of the system for correcting posture and method using the smart bag according to the embodiment of the present invention can be made. While the description of the present invention has been described with reference to specific embodiments, but various modifications are provided without deviating the scope of the present invention.

Although the present invention has been described by way of limited embodiments and drawings, the present invention is not limited by this and various modifications and variations can be made by those skilled in the art to which the present invention pertains. Those of ordinary skill in the art related to this embodiment will understand that it may be implemented in a modified form without departing from the essential characteristics of the above-described substrate.

Therefore, the disclosure methods should be considered from an explanatory point of view rather than a restrictive point of view. The scope of the present invention is shown in the claims rather than the foregoing description and all differences within the equivalent range should be interpreted as being included in the present invention.

1. A system for correcting posture comprising:
a smart bag including at least one pressure sensor and a first communication unit for transmitting an input value recognized by the pressure sensor to a user terminal; and
the user terminal including a second communication unit for receiving the input value from the first communication unit, an adhesion status checking unit for determining whether a user's human body and the smart bag are in close contact each other or not and determining an adhesion degree between the user's human body and the smart bag by using the input value received by the second communication unit, and a display unit for displaying the adhesion degree between the user's human body and the smart bag determined by the adhesion status checking unit,
wherein the user terminal further comprises:
a storage unit for storing a user's human body information, a posture and a length of an auxiliary strap matched to the user's human body information as well as the user's human body information; and
a control unit for generating a first control signal for winding or unwinding the auxiliary strap of the smart bag by using adhesion information between the user's human body and the smart bag,
wherein the second communication unit transmits the first control signal generated by the control unit to the first communication unit.

2. The system for correcting posture according to claim 1, wherein the user terminal includes an input unit for generating a second control signal to wind or unwind the auxiliary strap of the smart bag according to the user's intention,
wherein the second communication unit transmits the second control signal generated by the input unit to the first communication unit.

3. The system for correcting posture according to claim 1, wherein the user terminal includes:
an input unit for inputting an image obtained by capturing the user's human body and at least one of first to sixth input signals;
a posture determination unit for determining a posture of the user by using the image obtained by capturing the user's human body input by the input unit and at least one of the first to sixth input signals;
a storage unit for storing the lengths of the auxiliary strap matched to each posture of the user; and
a control unit for generating a third control signal to wind or unwind the auxiliary strap of the smart bag by using the lengths of the auxiliary strap matched to each posture of the user,
wherein the second communication unit transmits the third control signal generated by the control unit to the first communication unit.

4. The system for correcting posture according to claim 1, wherein the user terminal includes:
a winding unit for winding or unwinding the auxiliary strap so as to have a length corresponding to the first control signal received by the first communication unit; and
a motor unit for providing a driving force to the winding unit.

5. The system for correcting posture according to claim 2, wherein the user terminal includes:
a winding unit for winding or unwinding the auxiliary strap so as to have a length corresponding to the second control signal received by the first communication unit; and
a motor unit for providing a driving force to the winding unit.

6. The system for correcting posture according to claim 3, wherein the user terminal includes:
a winding unit for winding or unwinding the auxiliary strap so as to have a length corresponding to the third control signal received by the first communication unit; and
a motor unit for providing a driving force to the winding unit.

7. A method for correcting posture, comprising steps of:
transmitting an input value recognized by at least pressure sensor to a second communication unit by means of a first communication unit;
determining whether a user's body and a smart bag are in close contact or not, by using the input value received by the second communication unit, by means of an adhesion status checking unit;
determining an adhesion degree between the user's human body and the smart bag by means of the adhesion status checking unit; and
displaying the adhesion degree between the user's human body and the smart bag determined by the adhesion status checking unit by means of a display unit,
wherein the step of determining the adhesion degree between the user's human body and the smart bag by means of the adhesion status checking unit comprises steps of:
generating a first control signal for winding or unwinding an auxiliary strap of the smart bag by means of a control unit, by using adhesion information between the user's human body and the smart bag determined by the adhesion status checking unit;
transmitting the first control signal to the first communication unit by means of the second communication unit; and
winding or unwinding the auxiliary strap by means of a winding unit so as to have a length corresponding to the first control signal received by the first communication unit.

8. The method for correcting posture according to claim 7, wherein the step of determining the adhesion degree between the user's human body and the smart bag by means of the adhesion status checking unit comprises steps of:
generating a second control signal to wind or unwind the auxiliary strap of the smart bag according to the user's intention by means of an input unit;
transmitting the second control signal to the first communication unit by means of the second communication unit; and
winding or unwinding the auxiliary strap by means of the winding unit so as to have a length corresponding to the second control signal received by the first communication unit.

9. The method for correcting posture according to claim 7, wherein the step of determining the adhesion degree between the user's human body and the smart bag by means of the adhesion status checking unit comprises steps of:
inputting an image obtained by capturing the user's human body and at least one of first to sixth input signals by means of an input unit;
determining a posture of the user by means of a posture determination unit, by using the image obtained by capturing the user's human body and at least one of the first to the sixth input signals;
generating a third control signal to wind or unwind the auxiliary strap of the smart bag by means of the control unit, by using the length of the auxiliary strap matched to each posture of the user stored by a storage unit;
transmitting the third control signal to the first communication unit by means of the second communication unit; and
winding or unwinding the auxiliary strap by means of the winding unit so as to have a length corresponding to the third control signal received by the first communication unit.
